(19) United States
(12) Patent Application Publication
(10) Pub. No.: US 2011/0184412 A1
(43) Pub. Date: Jul. 28, 2011

(54) Pre-Assembled Construct With One or More Non-Rotating Connectors for Insertion Into a Patient

(75) Inventors: Christopher F Scifert, Bartlett, TN (US); Ian Rubin de la Borbolla, Memphis, TN (US)

(73) Assignee: WARSAW ORTHOPEDIC, INC., Warsaw, IN (US)

(21) Appl. No.: 12/695,595

(22) Filed: Jan. 28, 2010

(51) Int. Cl. A61B 17/56

(52) U.S. Cl. 606/60

(57) ABSTRACT

Surgical constructs pre-assembled for insertion as a unit into a patient. The construct may include an elongated member with opposing first and second ends and a non-circular cross-sectional shape. Connectors may be positioned on the elongated member and may include a first receptacle that receives the elongated member and a second receptacle spaced away from the first receptacle that receives a structural member in the patient. The first receptacles may be smaller than the elongated member to prevent the connectors from completely rotating around the elongated member. The construct may also include retaining members attached to the first and second ends of the elongated member. The retaining members may extend radially outward beyond the elongated member and may be larger than the first receptacles to prevent the connectors from escaping from the elongated member. Methods of assembling a construct prior to insertion into a patient are also disclosed.

### BACKGROUND

The present application is directed to a construct that is pre-assembled prior to being implanted into a patient to facilitate the insertion process, handling by the surgeon, and prevent detachment of one or more of the components.

Various different constructs are implanted into patients during surgical procedures. The constructs may connect to one or more elongated members, bone anchors, and various implant devices. The constructs often include multiple separate components that are attached together. Often times the components are physically small which makes them difficult to handle during the surgical procedure. The physical size may also make the components difficult to attach together during the surgical procedure. Further, the working area for the surgeon to implant the constructs is often relatively small. The small size may be the result of the patient anatomy and/or the type of construct being implanted into the patient. The working area may be further constrained by other surgical instruments that are within the patient at various times during the surgical procedure.

Some existing constructs include separate components that are attached together by the surgeon during the surgical process. This is often difficult for the surgeon because of the small size of the components and the relatively small working area.

### SUMMARY

The present application is directed to constructs that are pre-assembled prior to being implanted into a patient and methods of assembling the construct prior to implantation. The construct may include an elongated member with opposing first and second ends and a non-circular cross-sectional shape. Connectors may be positioned on the elongated member and may include a first receptacle that receives the elongated member and a second receptacle spaced away from the first receptacle that receives a structural member in the patient. The first receptacles may be smaller than the elongated member to prevent the connectors from completely rotating around the elongated member. The construct may also include retaining members attached to the first and second ends of the elongated member. The retaining members may extend radially outward beyond the elongated member and may be larger than the first receptacles to prevent the connectors from escaping from the elongated member.

A method of assembling a construct prior to insertion into a patient may include assembling together first and second connectors from a plurality of components. A first end of the elongated member may be inserted into a first receptacle of the first connector. The elongated member may include a non-circular cross-sectional shape. The first connector may be slid along an axial length of the elongated member while it is prevented from completely rotating around the elongated member. The second connector may be inserted on the elongated member and slid along the elongated member while it is prevented from completely rotating around the elongated member. The method may include attaching a first retaining member at a first end of the elongated member and a second retaining member at a second end of the elongated member with each of the first and second retaining members being larger than the first receptacles of the first and second connectors. The first connector may be secured at a first axial position along the elongated member, and the second connector may be secured at a second axial position along the elongated member. The pre-assembled construct may then be implanted into the patient.

The various aspects of the various embodiments may be used alone or in any combination, as is desired.

### BRIEF DESCRIPTION OF THE DRAWINGS

FIG. 1 is a schematic diagram of a construct according to one embodiment.

FIG. 2 is a perspective view of a pre-assembled construct according to one embodiment.

FIG. 3 is a side view of an elongated member according to one embodiment.

FIG. 4 is a sectional view taken along line IV-IV of FIG. 3 according to one embodiment.

FIG. 5 is a sectional view taken along line IV-IV of FIG. 3 according to one embodiment.

FIG. 6 is a sectional view taken along line IV-IV of FIG. 3 according to one embodiment.

FIG. 7 is a sectional view taken along line IV-IV of FIG. 3 according to one embodiment.

FIG. 8 is a sectional view taken along line IV-IV of FIG. 3 according to one embodiment.

FIG. 9 is a perspective view of a first end of an elongated member according to one embodiment.

FIG. 10 is a perspective view of a retaining member according to one embodiment.

FIG. 11 is a perspective view of a retaining member and a fastener according to one embodiment.

FIG. 12 is a perspective view of a retaining member according to one embodiment.

FIG. 13 is an exploded perspective view of a connector according to one embodiment.

FIG. 14 is a sectional view taken along line XIV-XIV of FIG. 13 that illustrates an assembled connector prior to placement on an elongated member.

FIG. 15 is a schematic end view of an elongated member within a first receptacle according to one embodiment.

FIG. 16 is a perspective view of a pre-assembled construct according to one embodiment.

### DETAILED DESCRIPTION

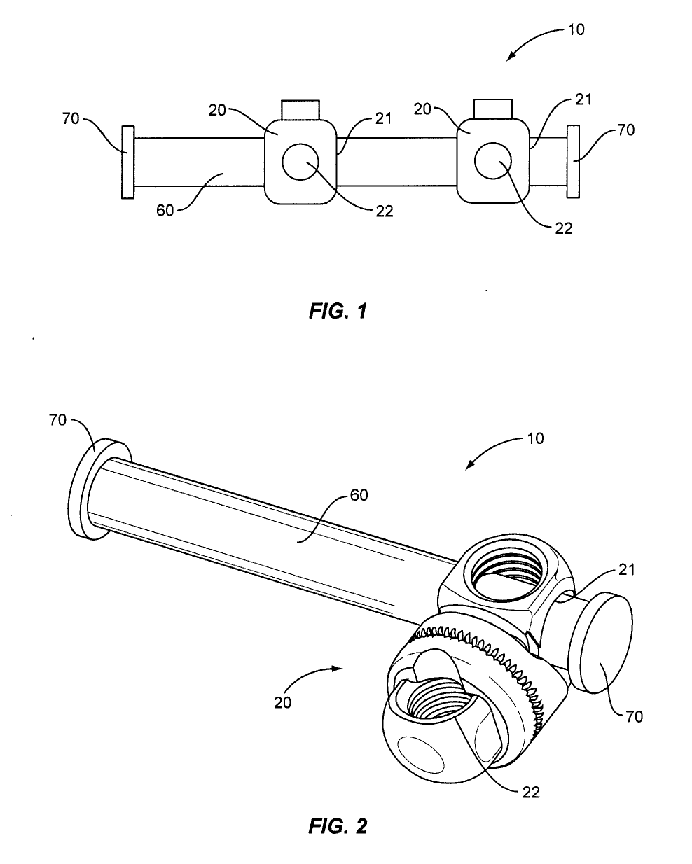

The present application is directed to a pre-assembled construct that may be inserted as a unit into a patient. FIG. 1 illustrates a construct 10 with an elongated member 60, a pair of connectors 20 attached to an elongated member 60, and a pair of retaining members 70. The elongated member 60 includes a non-circular cross-sectional shape that in combination with receptacles 21 in the connectors 20 prevents complete rotation of the connectors 20 around the elongated member 60. The retaining members 70 are positioned to maintain the connectors 20 on the elongated member 60. The pre-assembled construct 10 prevents the components from detaching during insertion into the patient and facilitates handling of the construct 10 by a surgeon. Preventing the connectors 20 from completely rotating around the elongated member 60 facilitates attaching a second receptacle 22 in the connectors 20 with one or more structural members in the patient.

FIG. 1 includes the pre-assembled construct 10 with a pair of connectors 20. Constructs 10 may include various numbers of connectors 20, including a single connector 20 as illustrated in FIG. 2. Constructs 10 may also include three or more connectors 20. FIG. 1 also includes a pair of retaining members 70. Other constructs 10 may include a single retaining member 70. In embodiments with multiple elements, the elements may be the same or may have a different size, shape, or construction.

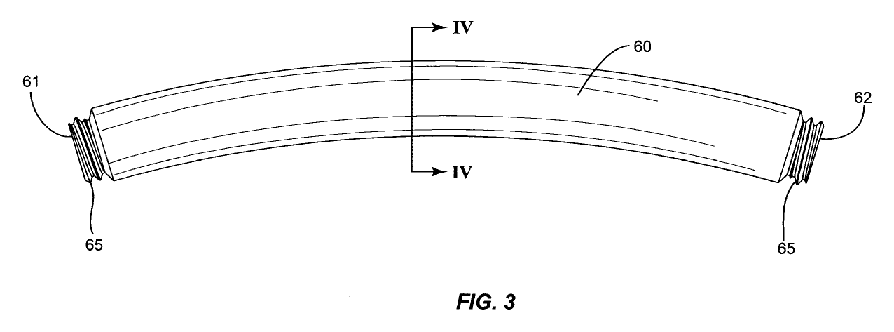

The elongated member 60 includes an elongated shape with a first end 61 and a second end 62 as illustrated in FIG. 3. The elongated member 60 may be substantially straight as illustrated in FIG. 2, or may be curved as illustrated in FIG. 3. The elongated member 60 may be curved along the entire length (i.e., from the first end 61 to the second end 62 as illustrated in FIG. 3), or may include one or more discrete curved sections.

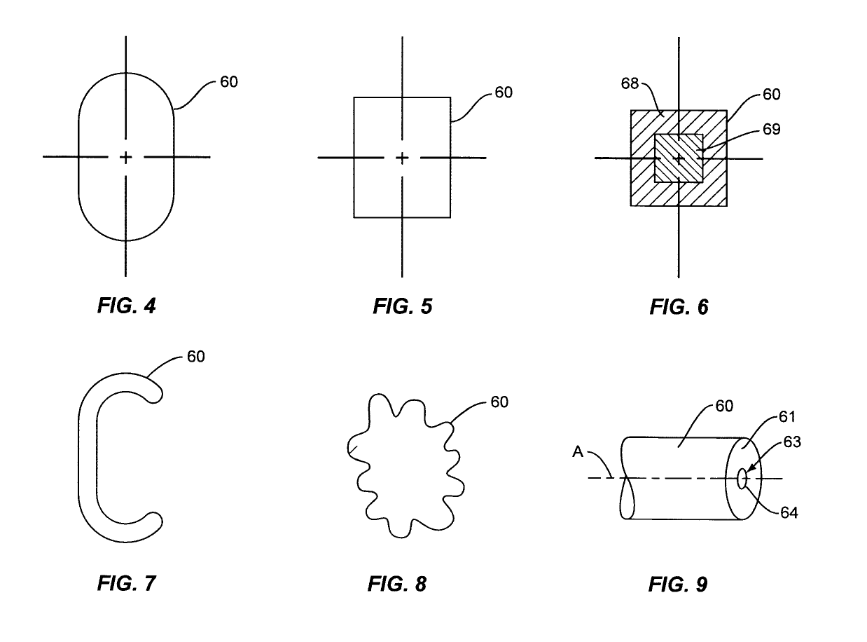

The elongated member 60 includes a non-circular cross-sectional shape. The shape and size of the elongated member 60 in combination with the receptacles 21 in the connectors 20 prevents the connectors 20 from completely rotating around the elongated member 60. The connectors 20 may be able to rotate a limited amount, but are prevented from complete rotation. The elongated member 60 may include a variety of different cross-sectional shapes. FIG. 4 includes an oval cross-sectional shape with a major axis Y and a minor axis X. Other non-circular cross-sectional shapes may include rectangular as illustrated in FIG. 5, square as illustrated in FIG. 6, an elongated channel as illustrated in FIG. 7, and a variable shape as illustrated in FIG. 8. Other embodiments may include one or more voids, a core of a different material, or a hollow core.

The elongated member 60 may be constructed from polyetheretherketone (PEEK). The elongated member 60 may also be constructed from other materials, including but not limited to plastic, stainless steel, titanium, cobalt chromium, ceramic, and combinations thereof. The elongated member 60 may include the same material construction throughout the cross-section as illustrated in FIGS. 4, 5, 7, and 8, or may include two or more sections of different materials as illustrated in FIG. 6.

The elongated member 60 includes connection features 63 at one or both ends 61, 62 to receive the retaining members 70. The connection features 63 are configured to receive a retaining feature 70. FIG. 9 includes an aperture 64 that extends into the elongated member 60 from the first end 61. The aperture 64 may extend a limited distance into the elongated member 60, or may extend the entire length. FIG. 9 includes the aperture 64 aligned along a longitudinal axis A of the elongated member 60. The aperture 64 may also be positioned away from the longitudinal axis A.

Another connection feature 63 includes threads 65 positioned at one or both ends 61, 62 of the elongated member 60 as illustrated in FIG. 3. The threads 65 extend inward a discrete distance from the ends 61, 62.

The elongated member 60 may include a connection feature 63 on just one end 61 or 62, or on both ends 61, 62. In embodiments with two connection features 63, the features 63 may be the same on each end 61, 62, or include different features on each end.

The retaining members 70 attach to the ends 61, 62 to maintain the connector 20 attached to the elongated member 60. In a preferred embodiment, the construct 10 includes a retaining member 70 at each end 61, 62 of the elongated member 60. In embodiments with multiple retaining members 70, the retaining members 70 may be the same or different. Other embodiments may include a single retaining member 70 at just one of the ends 61, 62. In one embodiment, the retaining members 70 are molded to the elongated member 60. The retaining members 70 may also be attached to the elongated member 60 inward from the ends 61, 62. The retaining members 70 may be constructed from a variety of materials, including but not limited to PEEK, titanium, titanium-alloys, cobalt chrome, stainless steel, and tantalum.

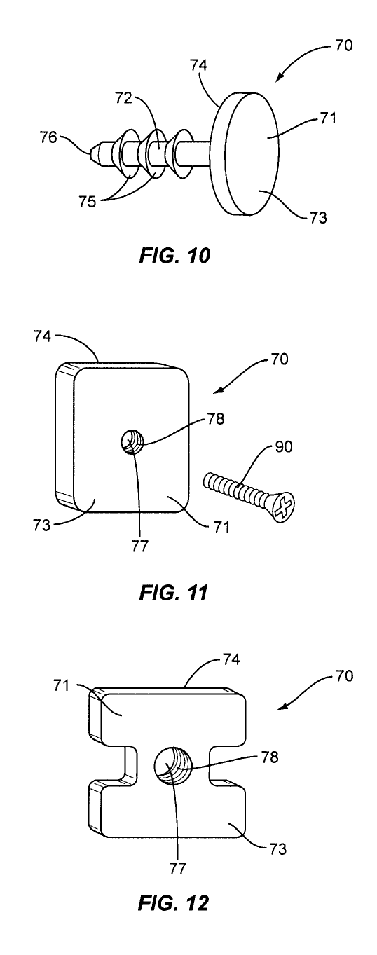

FIG. 10 includes a retaining member 70 that features a head 71 and a post 72. The head 71 is larger than the elongated member 60 and extends radially outward beyond the elongated member 60 when the retaining member 70 is attached to the elongated member 60. The head 71 may extend radially outward on all sides of the elongated member 60, or along a limited section. The head 71 is larger than the first receptacle 21 to maintain the connectors 20 on the elongated member 60 as will be explained in more detail below.

The head 71 may include various shapes, including a circular shape as illustrated in FIG. 10. The head 71 includes a first side 73 that faces away from the elongated member 60 and an opposing second side 74. The thickness of the head 71 measured between the sides 73, 74 may vary.

The post 72 extends outward from the second side 74 and attaches through an interference fit with the aperture 64 in the elongated member 60. The post 72 may include a variety of lengths and may include a tapered tip 76 to facilitate insertion into the aperture 64. One or more flanges 75 extend outward along the post 72. The flanges 75 include a larger size than the aperture 64 and deform when the post 72 is inserted into the aperture 64. The flanges 75 provide a stronger attachment between the retaining member 70 and the elongated member 60. A heat staking process may also be used to further enhance the attachment between the retaining member 70 and the elongated member 60. In another embodiment, the post 72 includes threads to attach within the aperture 64 in the elongated member 60.

FIG. 11 illustrates a retaining member 70 with an aperture 77 that aligns with the aperture 64 in the elongated member 60. A mechanical fastener 90 extends through the aperture 77 in the member 70 and into the corresponding aperture 64 to attach the retaining member 70 to the elongated member 60. The aperture 77 may include threads 78 that also engage with the threaded fastener 90.

FIG. 12 includes a similar embodiment with the aperture 77 sized to receive one of the ends 61, 62 of the elongated member 60. Threads 78 are configured to engage with threads 65 on the elongated member 60.

The various retaining members 70 may also be attached with additional means, such as but not limited to adhesives, heat staking, soldering, and brazing. In other embodiments, the retaining members 70 are attached with just these various means (e.g., adhesives) without any additional mechanical aspects. In these other embodiments, the second side 74 of the retaining member 70 may abut against one of the ends 61, 62 of the elongated member 60 to facilitate the attachment via these means.

The connectors 20 are attached to the elongated member 60 and are maintained by the retaining members 70. The connectors 20 include a first receptacle 21 to receive the elongated member 60 and are shaped to prevent the connector 20 from rotating completely about the elongated member 60. The connectors 20 also include a second receptacle 22 to attach the construct 10 to another structural element, such as a bone anchor that is attached to bony anatomy within the patient or another elongated member.

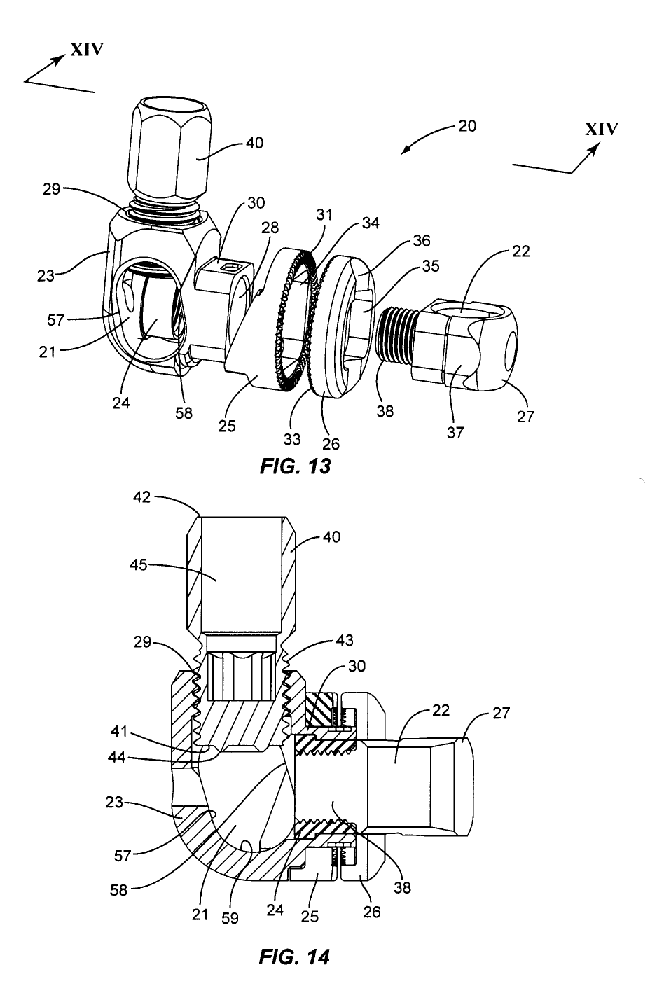

Various types of connectors 20 may be included as part of the construct 10. FIG. 13 includes an exploded view of a connector 20 that includes a first receptacle 21 to receive the elongated member 60 and a second receptacle 22 to receive the additional structural element. The connector 20 also includes a body 23 that includes the first receptacle 21, a fastener 40 that extends into the first receptacle 21, a nut 24, a pair of washers 25, 26, and a receiver 27 that includes the second receptacle 22.

The body 23 includes the first receptacle 22 that receives the elongated member 60, and first and second openings 28, 29 that extend into the first receptacle 21. The first opening 28 extends into the first receptacle 21 from a first direction and is formed within a neck 30 in the body 23. The second opening 29 extends into the first receptacle 21 from a second direction and is threaded to receive the fastener 40.

The fastener 40 fits within the second opening 29 and extends into the first receptacle 21 to contact against the elongated member 60. The fastener 40 includes a distal end 41 and a proximal end 42 and threads 43 that engage with corresponding threads in the opening 29. The distal end 41 may include one or more protrusions 44 with sharpened tips that contact against elongated member 60. The distal end 41 may also include a rounded, smooth face that contacts against the elongated member 60. The proximal end 42 may include a polygonal sectional shape to engage with an engagement tool for rotating the fastener 40 relative to the body 23. A cavity 45 may extend into the proximal end 42.

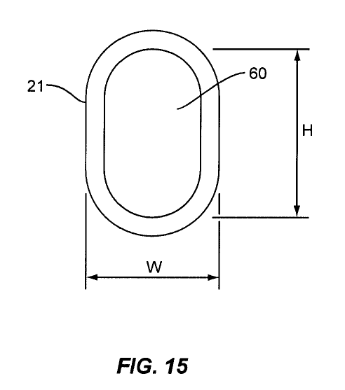

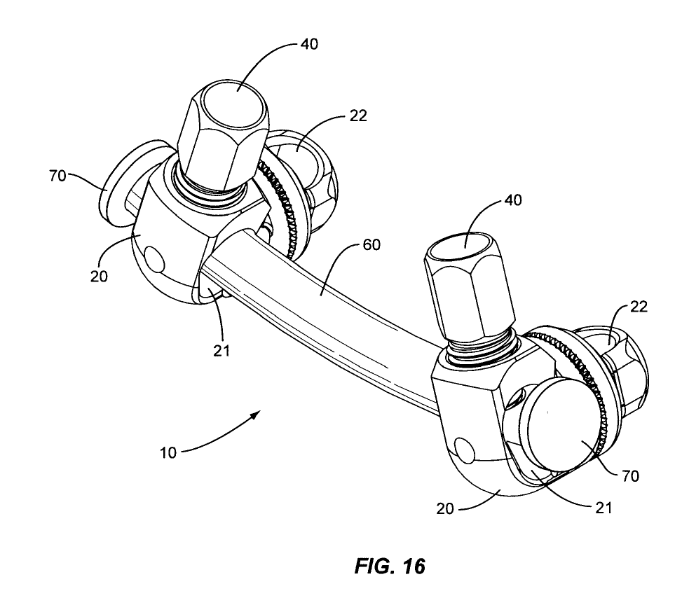

The first receptacle 21 is sized relative to the elongated member 60 for the connector to axially move along the elongated member 60. The first receptacle 21 is sized to allow some rotation around the elongated member 60 but prevent complete rotation around the elongated member 60. The first receptacle 21 includes at least one dimension that is smaller than the elongated member 60. FIG. 15 includes an embodiment with the width W of the first receptacle 21 being smaller than the height H of the elongated member 60. This sizing allows for some rotation of the connector 20 about the elongated member 60 but prevents the connector 20 from completely rotating about the elongated member 60. The partial rotation may facilitate alignment and attachment of the second receptacle 22 to the structural member in the patient.

The limiting dimension of the first receptacle 21 may include a fixed size, such as when the dimension is defined between opposing sidewalls of the first receptacle 21. An embodiment is illustrated in FIGS. 13 and 14 when the limiting dimension is fixed between opposing sidewalls 57, 58 of the first receptacle 21. Alternatively, the limiting dimension may be adjustable, such as when it is defined by the distal end 41 of the fastener 40 and the opposing sidewall 59 of the first receptacle 21, or by a component 25 of the connector 20 that extends receptacle 21 and the opposing sidewall 57. In these circumstances, the limiting dimension may be adjustable.

The connector 20 also includes the washers 25, 26 that each attach to the body 23 and are configured to adjust the angle of the second receptacle 22 of the receiver 27. The washers 25, 26 each include an opening 34, 35 respectively that align with the opening 28 in the body 23. The opening 34 in the first washer 25 may include a shape that matches the sectional shape of the neck 30. These corresponding shapes allow the first washer 25 to seat onto the neck 30 and prevent relative rotation. The first washer 25 may also be connected with an adhesive to the body 23. The opening 35 of the second washer 26 may correspond to the sectional shape of the receiver 27 as will be explained below. Splines 31, 33 are positioned on opposing faces and mate together at the desired angular position when the connector 20 is assembled together. The second washer 26 may also include a scalloped section 36 that faces outward towards the receiver 27. The scalloped section 36 conforms to the exterior shape of the structural member received in the second receptacle 22. The washer 25 may further extend onto the body 23 and into the first receptacle 21 to adjust the size of the first receptacle 21.

The receiver 27 includes the second receptacle 22 to receive the additional structural element, such as another elongated member or a bone anchor. The receiver 27 includes a main section 37 that forms the second receptacle 22 and an extension 38. The second receptacle 22 may include an elongated shape with a major axis that aligns with the openings 28, 34, 35, and a perpendicular minor axis. The extension 38 may be threaded and extend outward from the main section 37 and is received within the threaded nut 24. The sectional shape of the main section 37 may correspond to the sectional shape of the opening 35 for the main section 37 to seat within the opening 35 when the threaded extension 38 mates with the nut 24.

One example of a connector is disclosed in U.S. Pat. No. 7,261,715 herein incorporated by reference in its entirety. Other connectors include spinal systems TSRH 3D and TSRH 3Dx spinal available from Medtronic Inc., Spinal and Biologics, of Memphis, Tenn.

In use, the construct 10 is pre-assembled prior to insertion into the patient. The assembly may be completed at an off-site location (e.g., at the manufacturer) or at the time of the surgical procedure and prior to insertion. A benefit of the assembly at the time of the procedure is the ability to specifically tailor the construct to the specific needs of the patient. This may also reduce inventory of components as it may not be necessary to stock a variety of fully assembled constructs.

The construct 10 is assembled together prior to insertion into the patient. This may include assembling together one or more connectors 20 that are used in the construct 10. In embodiments with multiple connectors 20, the connectors 20 may be the same or may be different. Once the connectors 20 are assembled, they are attached to the elongated member 60. The attachment includes inserting one of the ends 61, 62 through each of the first receptacles 21 and sliding the connectors 20 along the elongated member 60. The connectors 20 are slid axially along the length while the relative sizes of the elongated member 60 and first receptacle 21 preventing complete rotation.

One or more retaining members 70 are attached to the ends 61, 62 of the elongated member 60 to maintain the attachment. Preferably, retaining members 70 are attached at each end 61, 62, although embodiments may include a single retaining member 70. The retaining members 70 include a larger size than the first receptacles 21 to maintain the connectors 20 on the elongated member 60 and prevent them from sliding off the ends 61, 62.

The construct 10 may be inserted into the patient with the connectors 20 still being axially movable along the elongated member 60 and partially rotatable. The connectors 20 may be positioned to engage with the other structural elements in the patient. Once positioned, the connectors 20 are secured in their axial and rotational positions by tightening the fasteners 40 into engagement with the elongated member 60. Alternatively, the connectors 20 are secured prior to insertion into the patient. FIG. 16 illustrates a pre-assembled construct 10 that is ready for insertion into the patient.

In one embodiment, the pre-assembled construct 10 is configured to connect to vertebral members. The elongated member 60 is a vertebral rod that has a length to extend along the desired vertebral levels. Prior to insertion of the construct 10, at least two bone anchors are attached to the vertebral members. Further, anchor posts are attached to each of the bone anchors and extend outward away from the vertebral members. The pre-assembled construct 10 includes a pair of connectors 20 each with a second receptacle 22 as illustrated in FIG. 16. The connectors 20 are positioned along the elongated member 60 with the second receptacles 22 aligning with and receiving the anchor posts. The anchors posts are inserted into the respective second receptacles 22 and the construct 10 is then moved along the anchor posts to the bone anchors. The retaining members 70 maintain the connectors 20 attached to the elongated member 60. In one embodiment, the connectors 20 are loosely positioned on the elongated member 60 and generally aligned with the anchor posts by the surgeons. Once aligned, the fasteners 40 are tightened to fix the position.

As illustrated in FIGS. 1, 2, and 16, the connectors 20 may be positioned adjacent to the retaining members 70 with no intervening elements. Therefore, the retaining members 70 alone function to maintain the attachment of the connectors 20 on the elongated member 60.

In one embodiment, the retaining members 70 are constructed of radio-opaque materials for post-operative viewing.

The constructs 10 may be implanted within a living patient or animal for the treatment of various spinal disorders. The constructs 10 may also be implanted in a non-living situation, such as within a cadaver, model, and the like. The non-living situation may be for one or more of testing, training, and demonstration purposes.

U.S. patent application Ser. No. 12/695,384 entitled “Pre-Assembled Construct for Insertion Into a Patient” is also directed to a pre-assembled construct and is herein incorporated by reference in its entirety.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A pre-assembled construct that attaches to structural members in a patient, the pre-assembled construct comprising:
an elongated member with opposing first and second ends and a non-circular cross-sectional shape;
a plurality of connectors positioned on the elongated member, each of the connectors including a first receptacle that receives the elongated member and a second receptacle spaced away from the first receptacle to receive one of the structural members in the patient, the first receptacles including a dimension that is smaller than the elongated member to prevent the connectors from completely rotating around the elongated member; and
retaining members attached to the first and second ends of the elongated member, the retaining members including a post that extends into an interior of the elongated member and a head that extends radially outward beyond the elongated member and is larger than the first receptacles to prevent the plurality of connectors from escaping from the elongated member.

2. The construct of claim 1, wherein the elongated member is made of PEEK.

3. The construct of claim 1, wherein the heads include a contact surface that faces towards and abuts against the elongated member with the posts positioned into the interior of the elongated member.

4. The construct of claim 1, wherein the cross-sectional shape of the elongated member is different than a shape of the head of the retaining members.

5. The construct of claim 1, wherein each of the plurality of connectors includes a fastener that extends into the first receptacle to contact against the elongated member and fix the axial position of the connector along the elongated member.

6. The construct of claim 1, wherein the dimension of each of the plurality of connectors is of a fixed size.

7. The construct of claim 1, wherein the cross-sectional shape of the elongated member is the same as each of the first receptacles.

8. A pre-assembled construct that attaches to structural members in a patient, the pre-assembled construct comprising:
a PEEK rod with opposing first and second ends and a non-circular cross-sectional shape;
a connector positioned on the elongated member and having a first receptacle that receives the elongated member and a second receptacle spaced away from the first receptacle to receive one of the structural members in the patient, the first receptacle including a fixed first dimension and an adjustable second dimension, the first receptacle being adjustable between a first size in which the connector can axially move along a length of the rod and is prevented from completely rotating around the rod, and a second size in which the connector is fixed axially on the rod and is prevented from rotating; and
a first retaining member attached to the first end of the rod and a second retaining member attached to the second end of the rod, the retaining members each being larger than the first receptacle in the first and second sizes to prevent the plurality of connectors from axially moving beyond the first and second ends of the elongated member.

9. The construct of claim 8, wherein the connector further includes a threaded fastener that extends into the first receptacle to adjust the second dimension.

10. The construct of claim 8, wherein the first retaining member includes a head that is larger than the first receptacle in the first size and an outwardly-extending post, the head abutting against the first end of the rod and the post positioned within an aperture that extends into the rod from the first end.

11. The construct of claim 10, wherein the second retaining member include a head that is larger than the first receptacle in the first size and an outwardly-extending post, the head abutting against the second end of the rod and the post positioned within an aperture that extends into the rod from the second end.

12. The construct of claim 10, wherein the post includes a plurality of flanges that are spaced apart along the post.

13. The construct of claim 8, wherein the first retaining member includes an aperture that aligns with an aperture in the first end of the rod.

14. The construct of claim 8, wherein the first dimension is defined between opposing sidewalls of the first receptacle and the second dimension is defined between a third sidewall of the first receptacle and a fastener attached to the connector.

15. The construct of claim 8, further comprising a second connector positioned on the elongated member, the second connector being substantially the same as the first connector.

16. A method of assembling a construct prior to insertion into a patient comprising:
moving first and second connectors onto an elongated member with a non-circular cross-sectional shape, the elongated member extending through first receptacles in each of the first and second connectors;
sliding the first connector along an axial length of the elongated member while preventing the first connector from completely rotating around the elongated member;
sliding the second connector along the axial length of the elongated member while preventing the second connector from completely rotating around the elongated member;
attaching a first retaining member at a first end of the elongated member and a second retaining member at a second end of the elongated member, each of the first and second retaining members being larger than the first receptacles of the first and second connectors;
securing the first connector at a first axial position along the elongated member;
securing the second connector at a second axial position along the elongated member that is spaced away from the first axial position; and
implanting the assembled construct into a patient.

17. The method of claim 16, further comprising assembling together the first and second connectors from a plurality of components.

18. The method of claim 16, wherein securing the first connector at the first axial position along the elongated member includes tightening a fastener on the first connector and contacting the fastener against the elongated member.

19. The method of claim 16, wherein preventing the first connector from completely rotating around the elongated member includes contacting an outer surface of the elongated member against an interior surface of the first receptacle.

20. The method of claim 16, wherein preventing the first connector from completely rotating around the elongated member includes connecting the elongated member against inner sidewalls of the first receptacle.
